(19) United States
(12) Patent Application Publication
(10) Pub. No.: US 2008/0109949 A1
(43) Pub. Date: May 15, 2008

(54) Swim goggles fastener system

(76) Inventor: Georgette U. Kinsella, Basking Ridge, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 11/590,096

(22) Filed: Oct. 31, 2006

(51) Int. Cl. A61F 9/02

(52) U.S. Cl. 2/426

(57) ABSTRACT

A swimming goggles fastener system includes a pair of swimming goggles having a slot at each end. Each slot is adapted to receive an inelastic retainer strap provided with adjacent Velcro hook and Velcro loop elements at opposing ends. One end of the inelastic strap is inserted into a slot of the swimming goggles and is folded, thereby engaging the Velcro hook and loop elements to attach the strap to the swimming goggles. The other end of the inelastic strap encircles the head of the swimmer. Thereafter, the other end is inserted into the other slot of the swimming goggle and folded to engage the Velcro hook and loop portions, securing the swimming goggles to the swimmer's head. Pressure applied to the swimmer's head by the swim goggles is adjusted by the Velcro hook and loop elements. Eyecup water proofing pads and optional rubberized compression cushion pads are attached to the inelastic strap to limit the pressure applied to a comfortable level. Advantageously, the swimming goggles resist displacement even under vigorous swimming activity such as competitive swimming, while reducing water drag on the swimmer, which would result from excessively protruding goggles.

### BACKGROUND OF THE INVENTION

1. Field of the Invention

The present invention relates to swimming goggles; and, more particularly, to a swim goggles fastener system having inelastic straps cushioned with rubberized foam material to provide an easily adjustable, tight, waterproof attachment mechanism that secures the swim goggles over the swimmers head while providing a comfortable feel, and resists displacement even under vigorous swimming activity such as competitive swimming, while reducing water drag on the swimmer that would otherwise be produced by excessively protruding goggles.

2. Description of the Prior Art

A number of patents disclose swimming goggles equipped with an attachment mechanism for securing the goggles to a swimmer's head. Oftentimes a rubber band is employed to attach the goggles to the head of a swimmer. This rubber band is all too readily dislodged and tends to result in a poor fit. It applies excessive pressure to the head portion of the swimmer, creating an unpleasant feel.

U.S. Pat. No. 3,710,393 to Douglas discloses a headgear. This semi-rigid head gear includes a bonnet to cover the hair portion. A transparent eye covering is created by two sheets of transparent material having a gap therebetween. A ventilating tube is used to remove the breath of the user. The elastic head band is optional. The headgear is designed for use when eye protection is needed, but is not used during swimming.

U.S. Pat. No. 3,755,819 to Douglas discloses a swimming headgear. This headgear has a nose-accommodating portion and eye-accommodating portions comprising an inner plate and an outer plate with a water filled channel therebetween to prevent fogging of the eye vision area. The nature of the water reservoir recess, especially at the temple area, permits upside down swimming substantially without the annoyance of water spilling out of the recess onto the eyes. The swimming gear is attached to the head of the user by a flexible elastic band. Due to the stretching characteristics of the elastic band, the headgear is not held securely in place. Adjustment of the headgear is not readily effected, since the elastic band is attached to the headgear by snap fasteners or buckles.

U.S. Pat. No. 4,162,542 to Frank discloses an eye protector. The eye protector comprises a three-piece device. Removable hemispherical eyecups are attached to each other by a transparent nose bridge, which permits tanning of the nose bridge. The eyecups may be tinted, transparent or opaque and the eye protector is used for sun tanning. Metallic plating of the cups keeps the cups cool when used in the direct sun. These eye cups are not used during swimming activities. No strap is used to attach the eye protector to the head of a wearer.

U.S. Pat. No. 4,176,410 to Matthias discloses a sports goggle. These sports goggles have a rectangular viewing area and are flexible so as to contour the head of the user. They are used for protecting eyes from wind and foreign particles during sporting activities such as skiing, cycling, auto road racing and the like. The sports goggles are attached to head of the user using flexible straps that are adhesively attached to the outer ends of the sports goggle. No disclosure is contained by the '410 patent concerning use of the sports goggles by swimmers.

U.S. Pat. No. 4,243,041 to Paul discloses cold pack goggles. A cold therapy pack is especially suited for use in the relief of pain, swelling, or other discomforts of patients involved in cosmetic surgery in the facial area such as, rhinoplasty and blepharoplasty. The appliance comprises a goggle shaped plastic pack formed from two layers of thermoplastic sheets filled with a hydrophilic gel having the property of maintaining its pliability in a frozen state. A separate nose pack is provided with a plurality of hydrophilic gel compartments. The cold pack goggle is secured around the head of a person using two separate detachable Velcro strips, the first strip being attached by cementation to slots in the goggles frame. One of the strips carries the hook portion of Velcro while the other strip carries the loop portion of the Velcro. Means are thereby provided for tightly attaching the goggle around the head of the user. No disclosure is contained by the '041 patent concerning use of a single non-elastic strap with both the hook portion of the Velcro and loop portion of a Velcro attachment means adjacent to each other on both sides of the strip. The cold pack goggles are not appointed for use by swimmers.

U.S. Pat. No. 4,686,712 to Spiva discloses a goggle mounting system. The goggle mounting system comprises protective goggles, which carry one strap on each side for attachment to a cyclist's helmet. The straps are flexible and resilient. Attachment of the straps to the helmet is such that the strap on only one side is easily disconnectable. The strap on the other side is pivoted to the helmet and remains permanently connected. The strap fasteners differ from one another in that one strap is attached to the helmet by means of mating sections of Velcro, respectively attached to the side of the helmet and the other side of the helmet is fixedly attached to the strap. A second strap is attached to the helmet by a resilient snap fixed to the helmet and a mating member on the end of the strap. The goggle is attached at one end to a helmet by a strap having a pivot snap. The other end of the goggle is attached to a strap that has a Velcro strip. The helmet has a Velcro portion to receive this Velcro strap attaching the goggle to the helmet. The goggle mounting system is not appointed for use with swimming goggles. It does not have a single non-elastic strap providing secure attachment of the helmet to the head of a wearer.

U.S. Pat. No. 4,790,031 to Duerer discloses an eye shield. A mask-like eye shield is used for sun bathing. The eye shield is made from soft fabric. A strap is optionally provided to attach the eye shield to the head of the user with an attachment means including Velcro, buckle, snap or other attachment means. The eye shield is made from ultraviolet transparent material providing tanning capability. It is not appointed for use in swimming goggles.

U.S. Pat. No. 5,042,094 to Sadowsky discloses eye wear with prosthetic parts for small children. Practical, safe and comfortable eye wear is appointed for use by babies and small children. A rigid shatterproof lens portion is encased in a padded and pliable frame that is strapped around the baby's head. Specially designed nose and temple pieces are incorporated into the frame to provide support for the frame on the baby's face in the absence of the normally supportive bone structure of adults. This is a tined shade worn by a child with a nose bridge and is attached by a strap that has Velcro on both sides. The frame of the glass has Velcro at both ends that mate with the Velcro of the strap. The strap is elasticized to insure comfort. It is not appointed for use in swimming goggles.

U.S. Pat. No. 5,046,200 to Feder discloses a reversible, quick-adjustable diver's face mask strap. A reversible, quick-adjustable diver's face mask strap includes a body portion formed of a flexible, elastic material having first and second opposite surfaces for contacting the head of a diver. The strap further includes first and second ribbons attached to the body portion, each of the ribbons having an attachment surface and another surface. Each of the ribbons has fasteners disposed on the attachment surface for releasably fastening the attachment surface to itself using hook and loop Velcro fasteners when folded to form a loop passing through an opening formed in a diver's face mask. One of the ribbons thereby remains adjustable without removing the mask strap from the diver's head regardless of which one of the opposite surfaces contacts the head of the diver. The elastomeric neoprene material of the strap is laminated with fabric formed of nylon, polyester or Lycra. The strap provides a low coefficient of friction on one side and has a high coefficient of friction on the other side. This strap attaches to a swimming goggle. The central portion of the strap is made from elastomeric neoprene and therefore stretches. The ends of the straps are connected to slots in the swimming goggle with one side facing inwards and other side facing outwards. The outward facing strap is used to adjust the swimming goggles' tightness. Since the central portion of the strap is elastic, the security of attachment of the swimming goggles is unreliable.

U.S. Pat. No. 5,213,241 to Dewar et al. discloses a ski goggle protective device. A protective device configured to accommodate snow ski goggles when the goggles are not to be used for vision purposes. This protector is placed over a ski goggle and strapped on to the biceps of the use for transporting a ski goggle. The protector has a central portion with two slots on its ends to receive a strap. The strap is stitched on one end, while the second end is fastened by Velcro hook and loop attachment. The protector is not a pair of swimming goggles. The strap does not surround the head of the user.

U.S. Pat. No. 5,309,577 to Buononato et al. discloses buoyant wrap-around sunglasses. The self-buoyant sunglass includes an elongated flexible strap having two free ends provided with fasteners joinable together to form a band, which encircles the head. This wrap-around sunglass has a strap extending from both ends of the sunglass and is attached to the head using a Velcro hook element attached to one end of the strap and a Velcro loop element attached to the other end of the strap. The buoyant wrap-around sunglasses are not appointed for use by swimmers.

U.S. Pat. No. 5,341,516 to Keim discloses a goggle support system. This protective goggle is worn by a fireman. The goggle has a nylon strap sewn in on one side and is connected to a main elastic strap by Velcro attachment. The goggle support system does not constitute a pair of swimming goggles. It uses elastic straps that are unreliable for energetic swimming activities.

U.S. Pat. No. 5,617,589 to Lacore et al. discloses combination headband, earcovers, and goggles. Winter sports goggles are secured by a two-sided headband made of a double layer of insulating fabric. Downward extensions of the fabric on the sides provide earcovers. The two sides of the headband overlap around the head and are adjustably connected by mating strips of hook and loop fasteners. The goggles are contoured with a foam edge contacting the face. Foam covered openings on the top and bottom edges of the goggles allow moisture to escape from behind the transparent windscreen, which filters ultraviolet rays. A narrow band of elasticized material sewn between the fabric layers of the headband connect each end of the headband to the goggles through a slotted extension from the goggles on each side or through slotted hooks snapping into openings in the sides of the goggles. The goggles are connected to a wide insulating strap that protects the ear when it wraps around the head of a user. The elasticized strap is tightened using a Velcro hook and loop connection. Straps on each end carry either the loop element or hook element of Velcro. This combination headband does not disclose or suggest a pair of swimming goggles.

U.S. Pat. No. 5,628,092 to Harris et al. discloses an eyewear retainer. The eyewear retainer has a strap that is attached to the earpieces of the eyewear and looped around the head of a human wearer to retain the eyewear snugly and securely in place on the wearer's head. The strap includes at least one hinge, a region of the strap having increased flexibility in comparison with a region adjacent to the hinge. The hinge is formed by pulling an end of the strap through an aperture through the strap's material. The strap may comprise two strap segments, each of which includes a hinge formed by passing an end of the strap segment through a separate aperture in the material of the strap segment. Two segment straps slip into the earpieces of an eye wear and are tightened against the user's head by Velcro attachment. The strap is elastic and carries either the hook or the loop portion of Velcro and mates with the other strap. The eyewear retainer would not be suitable for use during swimming activities.

U.S. Pat. No. 6,131,209 to Thayer et al. discloses an eyewear cleaning apparatus. Conventional eye glass ear pieces are inserted into an extended elastic tubular member to form an attachment with an elastic tubular member. When slipped over the head of a user, the attachment holds the eye wear against the eyes of the user. The tubular member has a compartment that carries a lens cleaning cloth. The compartment has Velcro closure flaps. This eyewear cleaning apparatus would not be suitable for use during swimming. The Velcro is not used to attach a strap to a goggle; but instead forms a closure mechanism for a lens cleaning cloth.

U.S. Pat. No. 6,952,841 to Schary et al. discloses sports goggles. These sports goggles are attached to the head of the user by two non extensible cords that connect to an elastic band. The presence of this elastic band contributes to a non-secure attachment of the sports goggles. The orientation of the goggles may be adjusted due to the elasticity of the strap. The sports goggles would not be suitable for use by swimmers.

U.S. Design Pat. D278,761 to Fuller discloses a waterproof belt container for a portable radio or tape player. The portable radio or tape player is contained in a slot, and the belt is attached to the wearer using a Velcro attachment mechanism. No disclosure is contained by the '761 patent concerning a pair of swimming goggles.

U.S. Design Pat. D294,710 to Witzel discloses a goggle strap having an ornamental design. The two ends of the strap have loops that are stitched together as indicated by the rectangular stitch line with cross stitches. It appears that the strap is inserted into slots in a goggle and is stitched together. The strap disclosed by the '710 design patent is not adjustable. It must be made from an elastic material to allow the goggle with this goggle strap to be inserted over the head of the user.

U.S. Design Pat. D330, 717 to Rupert et al. discloses a combined goggle strap and ear covers. This design patent discloses an ornamental design for a combined goggle strap and ear covers. The strap is wide enough to cover the ears of the user. The ends of the strap have a pair of Velcro strips designed for attachment to the slots of a pair of goggles. No disclosure is contained within the '717 design patent concerning the degree of elasticity of the strap.

U.S. Design Pat. D451,531 to Mabuchi discloses a pair of swimming goggles having an ornamental design. A foam rubber strap with a Velcro attachment on each of its ends is inserted into slots present in each side of a pair of swimming goggles. Since the rubber foam strap is expandable, the attachment of the swimming goggles to the swimmer is not tight and, due to the elasticity of the foam rubber strap, the goggle may migrate from one position to another during swimming.

U.S. Design Pat. D485,291 to Paolino discloses baby and toddler sunglasses having an ornamental design. The baby and toddler sunglasses have one end of a strap permanently attached to the sunglass. The other side of the strap is adjustable using a Velcro attachment mechanism. These sunglasses are designed for use by babies and toddlers. No disclosure is contained by the '291 patent concerning a pair of swimming goggles. In addition, there is no disclosure in the '291 patent as to whether the strap is elastic or non-elastic.

Foreign Patent Application DE3,524,392 to Deubel discloses a treatment mask. Application of cosmetic/pharmaceutical substances to the face is usually fairly troublesome. The invention describes a treatment mask, which is very easy to handle and thus facilitates treatment. At the same time, the treatment mask has a spectacle-like molding with retaining means and a carrier substance, which is in retentive connection with the spectacle-like molding and is filled with a pharmaceutically/cosmetically effective substance. The eye contacting portion is provided with mask treatment compositions and is held in place against the eyes of the user by two straps directly attached to the frame of the treatment mask. The two straps carry either a hook portion or a loop portion composed of Velcro material, for attaching the treatment mask over the head of a user. No disclosure is contained by the '392 foreign patent concerning a pair of swimming goggles. The strap is not a single piece, having a Velcro attachment mechanism on each of its ends.

Internet publication “EPG Ear Protector Goggles” at http://www.leisurepro.com/Prod/EPGC.html discloses EPG Ear Protector Goggles for Kids. The Ear Protective Goggles (EPG) cover the ears and prevent entry of water into ears during swimming. They incorporate swim goggles with a silicone-gasket ear seal. They are designed to keep water out of the eyes and ears while swimming. The EPG use a neoprene and Velcro strap system to incorporate an ear cover made of silicone that prevents water from getting into a swimmer's ear. The Ear Protective Goggles design allows the user to switch goggles from the ones supplied to their own personal favorite via two small Velcro goggle-fasteners. Since the neoprene and silicone strap attachment is elastic, the goggles will be displaced during vigorous swimming activity. Attachment of the goggles to the wide ear protecting strap is accomplished by a buckle, not a Velcro attachment mechanism.

Notwithstanding the efforts of prior art workers, swimming goggles which are retained on the head of a swimmer without displacement during vigorous competitive swimming or conventional relaxed swimming to thereby prevent entry of water adjacent to the eyes of the swimmer are as yet not available. Use of elastic straps fail to achieve this objective. These rubberized straps must be tightened with excessive tension, causing discomfort to the swimmer. There accordingly remains a need in the art for swimming goggles that are easily attached and adjusted, and resist displacement, remaining securely in place during vigorous competitive swimming or relaxed swimming activities. Also needed are swimming goggles that exert minimal tension on the head of the swimmer, thereby providing a comfortable fit.

### SUMMARY OF THE INVENTION

The present invention provides swimming goggles that have an inelastic strap equipped with Velcro hoop and loop elements on both sides thereof. The inelastic strap, together with the Velcro hook and loop portions on either side, is passed through slots provided in the left side and right side of the swimming goggles to easily and reliably secure the swimming goggles to the head of a swimmer. Since the strap is inelastic, it does not relax or change in length and therefore the fit of the swimming goggles is maintained and the swimming goggles are secure and free from any displacement. The pressure exerted by the swimming goggles strap on the swimmer's head remains precisely the same as that present when the swimming goggles were first secured on the swimmer's head. A competitive swimmer may choose to increase the pressure exerted by the strap due to the anticipation of vigorous swimming activity, while a relaxed swimmer may choose to reduce the pressure exerted by the swimming goggle strap. It is very difficult to apply extraordinary pressure to the head using the swimming goggle strap, since the eyecup portion of the swimming goggle is provided with a rubberized foam or other element that seals the eye cup against ingress of water. Accordingly, the inelastic strap provides a constant pre-selected pressure exerted at the eyecup, providing a reliable seal against penetration of water, which may distract a competitive swimmer.

The second embodiment of the invention incorporates one or more rubber strips in the inelastic strap of the swimming goggle. A single rubberized element may be provided at the center of the inelastic strip or several rubberized strips may be distributed along the length of the inelastic strip. Since the inelastic strip does not elastically stretch, the rubberized element is not stretched. It is merely compressed by the pressure applied to the head by the inelastic swimming goggle strip. For example, the rubberized element may be foam rubber, which can be compressed to a limited extent. The presence of one or more rubberized elements attached to the inelastic swimming goggle strap limits the overall pressure applied to the head of the swimmer regardless of the effectiveness of the eyecup sealing elements in limiting the applied pressure.

The swimming goggles to which the inelastic strap is attached are generally conventional in nature, except that the right and left ends of the goggles have a slot appointed to receive the Velcro ends of the inelastic strap. The swimming goggles may have glass or polymeric lenses which may be plain or tinted. The frame of the swimming goggles may be made from plastic, metal or other suitable material.

The swimmer first inserts the strap into the slot at the left or right side of the swimming goggles and closes the Velcro hook and loop sections to secure the strap on the swimming goggles frame. Thereafter, the user applies the swimming goggles, with the dangling strap, around the user's head and proceeds to insert the free end of the dangling strap into the right or left slot of the frame. The strap is then pulled and closed to engage the hook and loop portions of the Velcro attachment, securing the swimming goggle around the head of the swimmer with a customized degree of tightness or pressure, as selected by the user. If the eyecups are not positioned, the Velcro attachment straps at the left or right side are loosened and adjusted to bring the eyecups to a proper location. Once this adjustment has been made, a single user can leave the inelastic strap attached to the left or the right side of the swimming goggle frame, since it will come back to the previously selected position due to the inelastic nature of the strap. The eyecup seal elements are compressed by the strap, exerting sufficient pressure to block the entrance of water into the eyes of the swimmer, which can be annoying. This compression of the eyecup sealing element prevents application of excessive pressure to the head of the swimmer. In the second embodiment of the invention, one or more additional rubberized elements are permanently attached to the inelastic strap, restricting application of excessive pressure to the swimmer's head by the inelastic swimming goggle strap.

### BRIEF DESCRIPTION OF THE DRAWING

The invention will be more fully understood and further advantages will become apparent when reference is had to the following detailed description of the preferred embodiments of the invention and the accompanying drawings, in which:

FIG. 1 is a schematic diagram depicting the swim goggles fastener system;

FIG. 2a illustrates the inelastic strap of the swim goggles fastener system according to the first embodiment of the invention;

FIG. 2b illustrates the inelastic strap of the swim goggles fastener system according to the second embodiment of the invention; and

FIG. 2c illustrates the inelastic strap attached to the swimming goggles with the length of the inelastic strap properly adjusted.

### DETAILED DESCRIPTION OF THE INVENTION

The present invention provides a swim goggles fastener system that stays in place even during vigorous swimming activity. This functionality is accomplished by using a non-elastic strap member equipped with Velcro™ hook and loop attachments at each of its ends. The non-elastic strap is inserted into a pair of swimming goggles having a slot provided at each end thereof. The Velcro™ hook and loop attachment enables the adjustable attachment of the strap providing a tightness feel that is comfortable to the user. At the same time, the adjustable attachment of the strap securely holds the swimming goggles in place on the user's head throughout the time that the system is worn. The swimming goggle has a rubberized elastic sealing element at the eyecup-contacting area, waterproofing the swimming goggles. The presence of this elastic sealing element provides some cushioning effect and prevents excessive tightness to the user's head as the swimming goggle is secured in place over the head of the user. In the second embodiment of the invention, additional rubberized compressive pads are permanently attached to the inelastic strap to limit the pressure applied to the head of the swimmer. The Velcro™ hook and loop attachment also prevents excessive tightness due to the loose interaction between the hook and loop elements of the Velcro strip attachment. At any time, the user may tighten or loosen the Velcro™ hook and loop attachment. The System thereby provides for easy adjustability of the swimming goggles. During competitive swimming events, the swimming goggles may be tightened sufficiently to prevent dislodgement during critical, highly energetic competitive activities, such as swim meets, water polo, and the like.

The non-elastic strap may be fabricated from a number of materials including cotton, wool, rayon or other natural materials or synthetic materials including nylon, polyester or polyolefins. These fibers may also be combined to produce the non-elastic strap.

Since the strap is non-elastic; the Velcro™ hook or loop fasteners can be readily attached to the strap by sewing, adhesive bonding or other suitable methods. Generally, the backing material for a Velcro™ strip does not stretch and cannot be easily attached to elastic strips since there is a large discontinuity in the deformation under load properties of Velcro™ strip and elastic strap, thereby producing enormous stresses at the bond region essentially delaminating or breaking the bond. This reduces the useful shelf life and reliability of swimming goggles having an elastic strap with a Velcro™ attachment mechanism. By way of contrast, these attachment problems are virtually eliminated when the strap is non-elastic. Accordingly; this inelastic property represents a key feature of the subject invention.

Generally stated, the Swim Goggles Fastener System is adapted to act as a head strap for swim goggles with the strap being non-elastic, and held in place by a pair of Velcro™ attachment means. The Swim Goggles Fastener System provides a quick and easy head strap adjustment and stays in place without expansion due to the non-elastic nature of the strap. The Velcro™ hook and loop fastener utilized by the head strap is waterproof. The Swim Goggles Fastener System allows greater streamlining of the goggles by allowing the user to readily adjust the head strap for optimal comfort without the need for head strap clips. As a result, the goggles can be adjusted to fit the user's head in a snug manner, causing the goggles to be held securely on the swimmer's head during swim races. On the other hand, during recreational usage of the swim goggles, the Swim Goggles Fastener System is readily adjusted to achieve a less snug fit. This, in turn affords greater comfort to the wearer.

In use of the first embodiment of the invention, the inelastic strap is initially inserted into the slot of the swimming goggles frame, and the Velcro elements are closed to secure the strap thereto. The dangling inelastic strap is then secured in place around the head of the swimmer at a selected convenient location. The free end of the dangling inelastic strap is inserted into the slot on the opposite side of the swimming goggles frame and the Velcro hoop and loop portions at the inserted end are closed to secure the swimming goggle in place against the head of the swimmer. If the eyecups are not in a proper position, the Velcro attachment at either end of the inelastic strap can be adjusted to retain the eyecups at the proper location, are a pre-selected pressure that depends on the level of swimming activity. Generally, a competitive swimming event will require a higher pressure, as compared to a relaxed swimming activity, to prevent entry of water into the eyecups.

In accordance with the second embodiment of the invention, the inelastic strap is provided with one or more rubberized compression cushions permanently attached to the inelastic strap. Since the inelastic strap does not extend in length, the rubberized cushion elements are not stretched. They are merely compressed by the pressure that is present when the inelastic strap is tightened against the swimmer's head. The compressibility of the rubberized compression cushion pads effectively limits the pressure applied to the swimmers head when the inelastic strap is tightened.

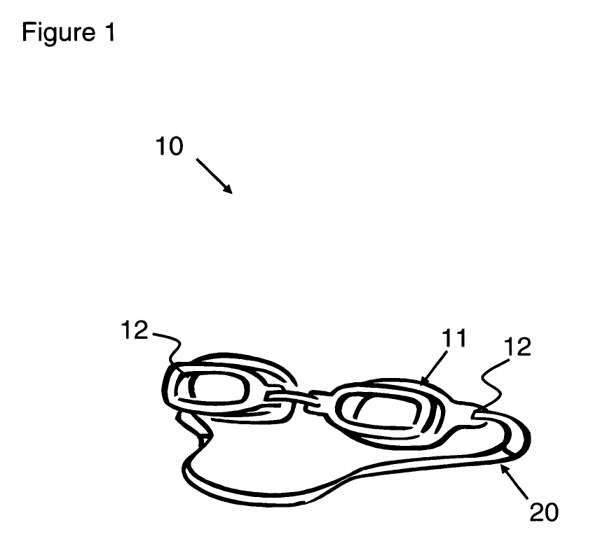

Referring to FIG. 1, there is shown generally at 10, a swim goggles fastener system attached to a pair of swim goggles. The swim goggles 11 include side portions 12 having an opening or a slot adapted to receive a non-elastic strap at each end. Inelastic head strap 20 is received in the openings of side portions 12 so that the swim goggles 11 can be adjusted to fit the user's/swimmer's head.

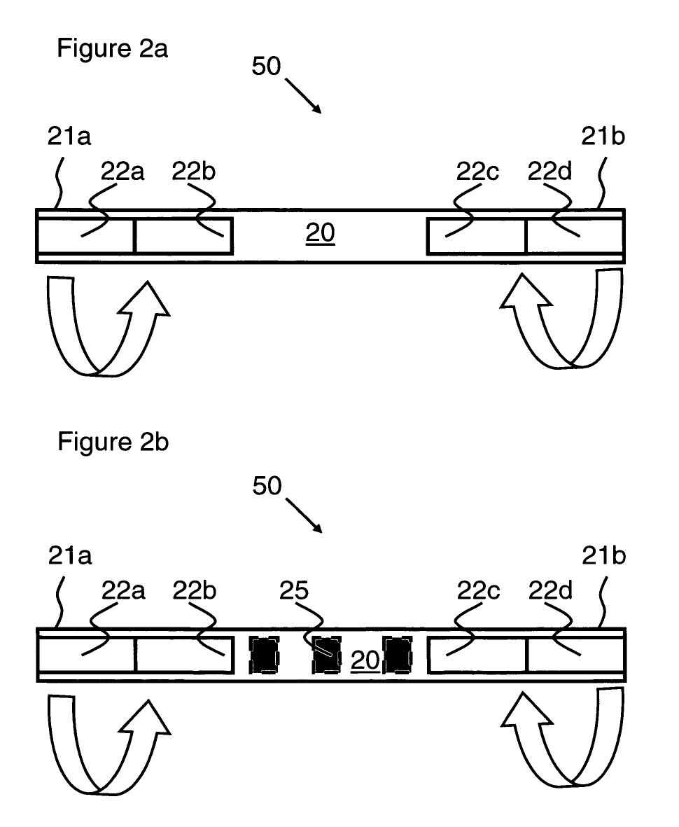

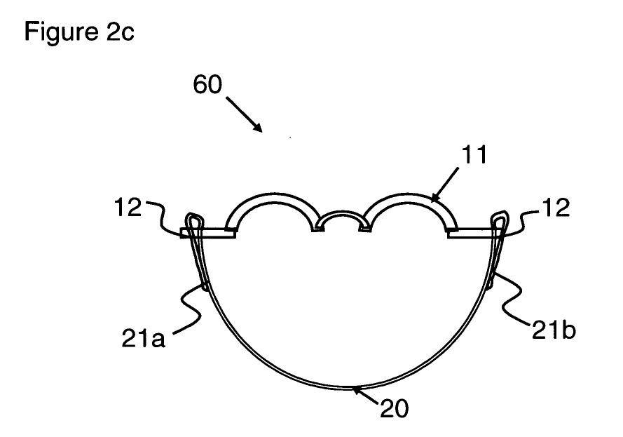

FIGS. 2a and 2b illustrate the inelastic strap of the swim goggles fastener system according to the first and second embodiment of the invention.

Specifically, in FIG. 2a there is shown generally at 50 a top planer view of inelastic strap 20 of the swim goggles fastener system when it is not attached to a pair of swimming goggles. The inelastic strap is a flat non-elastic strip with Velcro™ hook and loop strips attached at each end, as shown. Inelastic head strap 20 includes a first end 21a and a second end 21b. First end 21a of the inelastic strap 20 further comprises a first Velcro portion with hook portion 22a and loop portion 22b. Second end 21b of the inelastic strap 20 further comprises second Velcro portion with hook portion 22c and loop portion 22d.

FIG. 2b shows generally at 50 a top planer view of the inelastic strap 20 of the swim goggles fastener system when it is not attached to a pair of swimming goggles, according to the second embodiment of the invention. The inelastic strap is a flat non-elastic strip with Velcro™ hook and loop strips or portions attached at each end, as shown. Inelastic head strap 20 includes a first end 21a and a second end 21b. First end 21a of the inelastic strap 20 further comprises a first Velcro portion with hook portion 22a and loop portion 22b. Second end 21b of the inelastic strap 20 further comprises a second Velcro portion with hook portion 22c and loop portion 22d. The inelastic strap 20 has one or more (three shown in this illustration) rubberized compression pads 25, shown as dashed lines since the rubberized compression pads are affixed to the side of the inelastic strap that is opposite to the side having the Velcro hook and loop elements.

FIG. 2c shows generally at 60 a top view of the head strap 20 of the swim goggles fastener system, wherein the inelastic head strap 20 is attached to swim goggles 11. The swim goggles fastener system comprises inelastic strap 20. As is illustrated by the arrows in FIG. 2a and illustrated by way of FIG. 2b, each of the first and second ends 21a and 21b of the head strap 20 are adapted to be received by ends of a swim goggle 11, so that swim goggle 11 can be readily adjusted to fit a user. Each of the first and second ends 21a and 21b of the head strap 20 include the first and second Velcro portion pairs 22a, 22b and 22c, 22d. First end 21a and second end 21b of head strap 20 slide through slots on the sides portions 12 of swim goggles 11 and attach to the incoming portions of the first and second Velcro portion pairs 22a, 22b and 22c, 22d of the head strap 20 as it is pulled out for adjustment, as shown by the two positions of the strap 20.

The swim goggles fastener system comprises the following features, in combination:
1) swim goggles provided with slots on both sides adapted to receive an adjustable swim goggles retaining strap;
2) the swim goggles having rubberized cushion pads at eye contacting areas thereof;
3) the retaining strap being composed of a non-elastic material including natural materials or synthetic materials;
4) the natural materials for the non-elastic strap comprising cotton, wool, rayon and the like;
5) the synthetic materials for the non-elastic strap comprising nylon, polyester, olefin and the like;
6) the non-elastic strap comprising Velcro™ hook and loop strips at first and second ends thereof, and having a hook end portion and mating loop end portion appointed for disposition adjacent to each other at said ends;
7) the first hook end of the non-elastic strap being inserted into a slot at one end of the swim goggles and folded to mate with the adjacent loop portion at the first end, to thereby attach the non-elastic strap to the swim goggles at one end;
8) the second hook end of the non-elastic strap being inserted into a slot at the other end of the swim goggles and folded to mate with the adjacent loop portion at the second end, to thereby attach the non-elastic strap to the swim goggles at the other end of the swim goggles;
9) the length of the non-elastic strap being adjusted by opening out and then tightening or loosening the Velcro attachment at either end to snugly fit the head of the user;
10) the retaining strap optionally having one or more rubberized compression cushions attached thereto which do not stretch but are compressed when the retaining strap is tightened against the head of the swimmer;
11) the rubberized cushion pads at the eyecup region and the non-elastic strap holding the swim goggles firmly against the eyes of the user, providing a water tight seal;
12) the rubberized cushion pads and one or more rubberized compression cushions limiting the pressure exerted by the inelastic strap against the head of the swimmer;
whereby the swim goggles are readily adjusted to provide a snug fit or a relaxed fit, depending on user preferences and the nature of an appointed swimming event, and the swim goggles resisting displacement even during energetic swimming events such as time trials and swim team competitions.

Having thus described the invention in rather full detail, it will be understood that such detail need not be strictly adhered to, but that additional changes and modifications may suggest themselves to one skilled in the art, all falling within the scope of the invention as defined by the subjoined claims

What is claimed is:

1. A swim goggle fastener system comprising:
a. a pair of swim goggles provided with slots at each end adapted to receive an adjustable swim goggles retaining strap;
b. said swim goggles having rubberized eyecup cushion pads contacting the face of a swimmer, preventing entry of water into the eyes during swimming;
c. said retaining strap being composed of inelastic strap material;
d. said inelastic retaining strap comprising a Velcro hook portion and mating Velcro loop portion adjacent to each other at each end;
e. said Velcro portion of one end of said inelastic strap being inserted into a slot of said swimming goggle and folded to engage said hook and loop and secure said inelastic strap to said swimming goggle at one end with the free end dangling;
f. said dangling unattached portion of said inelastic strap being adapted to be wrapped around a swimmer's head and inserted into a slot of said swimming goggles and folded to engage the hook and loop portions of the Velcro when attaching the swimming goggles to the swimmer's head;
g. the position of said eye cup cushions and the pressure on the swimmer's head being adjusted by positioning the Velcro attachments at each end of the swimming goggles;
whereby said eyecup cushions limit pressure on the swimmer's head and the inelastic strap retains the swimming goggle in place without displacement even during vigorous swimming activity.

2. A swim goggle retainer system as recited by claim 1, wherein said inelastic strap is selected from natural materials, synthetic materials, or combinations thereof.

3. A swim goggle retainer system as recited by claim 2, wherein said natural materials comprise cotton, wool, rayon and combinations thereof.

4. A swim goggle retainer system as recited by claim 2, wherein said synthetic materials comprise nylon, polyester, olefin and combinations thereof.

5. A swim goggle fastener system comprising:
a. a pair of swim goggles provided with slots at each end adapted to receive an adjustable swim goggles retaining strap;
b. said swim goggles having rubberized eyecup cushion pads contacting the face of a swimmer, preventing entry of water into the eyes during swimming;
c. said retaining strap being composed of inelastic strap material;
d. said inelastic retaining strap comprising a Velcro hook portion and mating Velcro loop portion adjacent to each other at each end;
e. said inelastic retaining strap comprising one or more rubberized compression cushion pads permanently attached thereto;
f. said Velcro portion at one end of said inelastic strap being adapted to be inserted into a slot of said swimming goggle and folded to engage said hook and loop portions and secure said inelastic strap to said swimming goggles at one end, with a free end dangling;
g. said dangling unattached portion of said inelastic strap being adapted to be wrapped around a swimmer's head and inserted into a slot of the swimming goggles and folded to engage the Velcro hook and loop portions to thereby attach the swimming goggles to the swimmer's head;
h. the position of said eye cup cushions and the pressure on the swimmer's head being adjusted by positioning the Velcro portions at each end of swimming goggles;
whereby said eyecup cushions and the rubberized compression cushion pads limit the pressure on the swimmer's head and the inelastic strap retains the swimming goggles in place without displacement even during vigorous swimming activity.

6. A swim goggle retainer system as recited by claim 5, wherein said inelastic strap is selected from natural materials, synthetic materials, and combinations thereof.

7. A swim goggle retainer system as recited by claim 6, wherein said natural materials comprise cotton, wool, rayon and combinations thereof.

8. A swim goggle retainer system as recited by claim 6, wherein said synthetic materials comprise nylon, polyester, olefin and combinations thereof.

9. A swim goggle retainer system as recited by claim 5, wherein said rubberized compression cushion pad is a foam rubber pad.
